(19) United States
(12) Patent Application Publication
(10) Pub. No.: US 2009/0052184 A1
(43) Pub. Date: Feb. 26, 2009

(54) Multi-Purpose Light Source

(75) Inventors: Robert Ibsen, Santa Maria, CA (US); John West, Arroyo Grande, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Holdings LLC, Santa Maria, CA (US)

(21) Appl. No.: 12/167,677

(22) Filed: Jul. 3, 2008

Related U.S. Application Data
(60) Provisional application No. 60/929,661, filed on Jul. 6, 2007.

(51) Int. Cl. F21V 9/00; A61B 18/18; A61N 5/06

(52) U.S. Cl. 362/293; 606/13; 607/88; 433/215

(57) ABSTRACT

The present invention is a multi-purpose light source of a unique design and specialized attachments which are also independently unique that can be used for, but not limited to, Dental, Medical, Cosmetic, and Industrial applications and procedures. Specifically the spectral irradiance of the light source can be controlled in such a way as to allow it to be used for procedures currently performed by lasers, electrosurgical devices, and hand instruments while retaining the benefits of the light source for other uses such as the photo-initiation of resins, tooth whitening, fluorescence, and illumination. The present invention may be used instead of a laser either independently or in conjunction with electrosurgical devices and hand instruments.

This application claims the priority of U.S. Provisional Application No. 60/229,661 filed on Jul. 6, 2007, the disclosure of which is expressly incorporated by reference herein.

### FIELD OF THE INVENTION

The present invention relates generally to light sources for use in dental and medical procedures.

### BACKGROUND OF THE INVENTION

Current cosmetic and surgical soft tissue procedures are performed with hand instruments like a scalpel, electrosurgical instruments, and lasers. Although instruments like a scalpel are the standard for surgical precision there is no hemostasis. Electrosurgical instruments provide hemostasis but are not as precise as hand instruments or lasers. Lasers can be used like a hand instrument to ablate, incise, excise, resect, dissect, or amputate tissue with contact fiber providing tactile feedback. Hemostasis provides clear field at the target site. Cutting with lasers is slower than hand instruments but provide better access to tissue in confined areas. Lasers have the disadvantage of being monochromatic and are expensive. The selection of wavelengths for diode lasers currently used for cosmetic and surgical soft tissue procedures is not based on the maximum absorption wavelength of target tissue. A very limited number of wavelengths are currently available with enough power to have the desired effect on tissue. High power laser diodes were primarily developed to optically pump (excite) solid-state lasers into stimulated emission. Typical wavelengths are 808 nm, 810 nm, 830 nm, and 980 nm. None of these wavelengths match the peak absorptive region of soft tissue targets such as hemoglobin, melanin, and water. Water is especially important in light-tissue interactions because it is prevalent in significant amounts in all tissue. Light energy that is absorbed by water molecules is converted to heat and provides a reliable method of heating adjacent tissue. Hemoglobin and melanin may not always be present to such a high degree to act as a heat conductor. Because the laser diodes do not closely match the peaks of targets the desired tissue interaction takes longer and/or power levels need to be increased. As power levels are increased heat transfer to non-target tissues and increased penetration depth become a concern.

Laser diodes with wavelengths of 808 nm to 980 nm fall at the low end of the absorptive region of water. Light wavelengths above 1 micron (1000 nm) are more readily absorbed by water. Xenon, and other, lamps emit a broad spectrum of light energy that can be filtered to transmit wavelengths that more closely match the peak absorptive region of targets. The xenon lamp of the present invention emits electromagnetic energy at a much higher point of the absorptive curve of water and matches the peaks of hemoglobin and melanin. Higher absorption enables the xenon light source to work more quickly and efficiently causing less chance of collateral damage through hear transfer to non-target tissue.

One of the problems associated with using xenon or other types of lamps for “laser” procedures is getting enough light energy into and out of a light guide with a very small diameter. Laser diodes are electrically very efficient and can convert as much as 50% of the electrical input power into light output. Laser light is collimated so that it can enter a single fiber typically from 100-400 microns. Xenon lamps are not as efficient and the light is emitted from the lamp at very steep angles. The xenon lamp of the present invention and optical characteristics of the light guide make it possible to deliver light energy levels similar to other lasers currently on the market.

Use of lasers is highly regulated because of safety issues. Many states regulate lasers and hygienist, dental assistants, cannot use them and nurses who need to use them the most for hygiene and therapy procedures.

There are numerous dental procedures today that require a source of electromagnetic radiation. Some examples of these light producing instruments used in the typical dental practice include; resin light curing units, peroxide tooth whitening systems, soft and hard tissue lasers, caries detection, oral tissue examination, tooth transillumination, tooth color matching, and illumination of the oral cavity. Additional uses of light in the dental practice may include bio-stimulation, pain relief, and other surgical and therapeutic indications.

In order to offer the benefits of these instruments, the practitioner must purchase each unit individually with most costing several thousands of dollars each. Often, when faced with financial constraints, the practitioner must decide against one or more of these instruments thereby reducing the quality of care provided to the dental patient.

The purpose of the present invention is to provide one source of electromagnetic radiation that can emulate the characteristics of one or more of a group of electromagnetic radiation generating instruments.

### SUMMARY OF THE INVENTION

The present invention is a multi-purpose light source of a unique design and specialized attachments which are also independently unique that can be used for, but not limited to, Dental, Medical, Cosmetic, and Industrial applications and procedures. Specifically the spectral irradiance of the light source can be controlled in such a way as to allow it to be used for procedures currently performed by lasers, electrosurgical devices, and hand instruments while retaining the benefits of the light source for other uses such as the photo-initiation of resins, tooth whitening, fluorescence, and illumination. The present invention may be used instead of a laser either independently or in conjunction with electrosurgical devices and hand instruments.

One embodiment of the present invention comprises a multi-use light comprising: a light source; a light guide; a filter changer; and a power supply.

Another embodiment of the instant invention is directed to a method of modifying the output of a light source comprising: providing a source of light; guiding the light; modifying the wavelength of the light by passing through a filter; and directing the modified light to a desired target.

A further embodiment of the present invention is directed to a multi-use light comprising: a light source; a means for guiding light; a means for filtering light; and a means for supplying power.

A further embodiment of the present invention is drawn to a method of treating tissue comprising providing a source of light; guiding the light; modifying the wavelength of the light by passing through a filter; and directing the modified light to a desired tissue.

### BRIEF DESCRIPTION OF THE FIGURES

FIG. 1 shows the broad spectral output of the light source of one embodiment of the present invention before filtration.

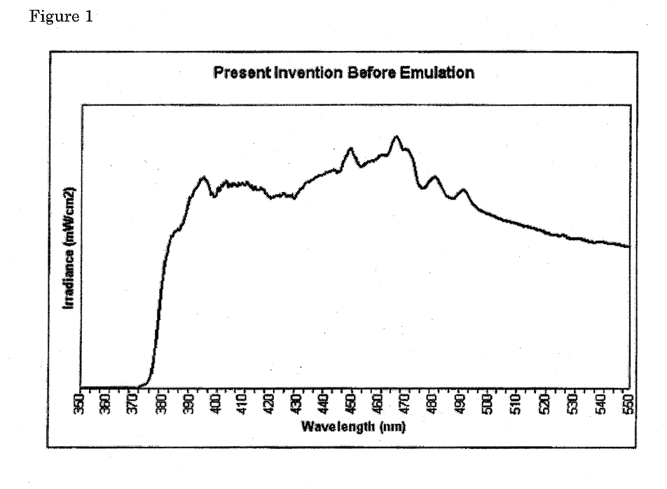

FIG. 2 shows the spectral output after the light has been filtered in one embodiment of the present invention.

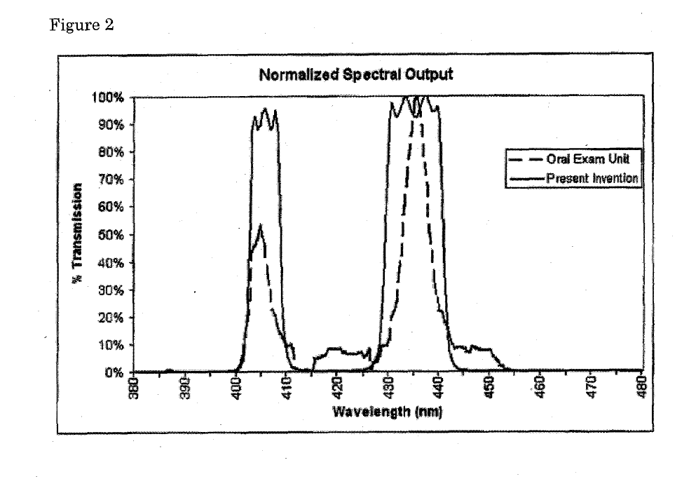

FIG. 3 shows the broad spectrum of light produced by an embodiment of the instant invention.

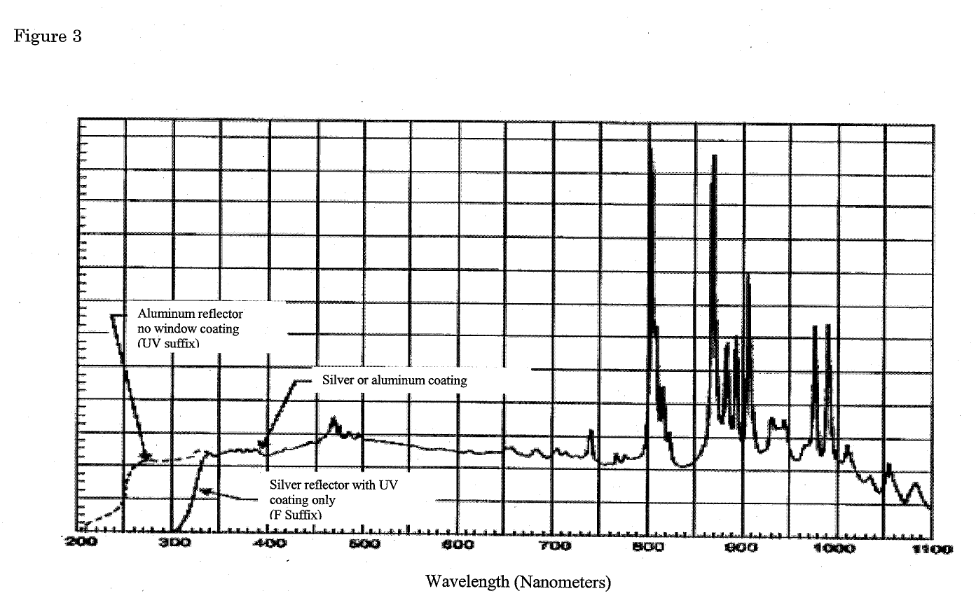

FIG. 4 shows the absorptive ranges of different biological tissues and organic matter.

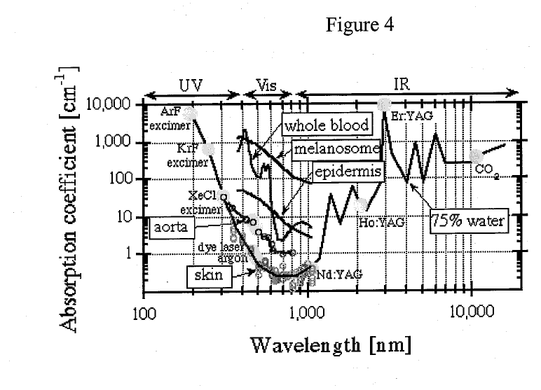

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention generates electromagnetic energy that is controlled and delivered by unique optical, electronic, and electromechanical devices for the purpose of producing predictable effects on biological tissue, photo initiation of dental light cure resins and other light cure materials, activation of tooth whitening agents, and illumination. Indications of use are for, but not limited to, Dental, Medical, Cosmetic, and Industrial applications and procedures. The present invention can be used to perform procedures that currently require the use of a laser while retaining the benefits of photo initiation of resins, tooth whitening, fluorescence, and illumination.

The present invention may be used in dental and medical procedures where light interaction with biological tissue through an optical component less than 1 mm in diameter or larger is desired. The present invention may be used for the photo initiation of dental light cure resins or other light cure materials through an optical component less than 1 mm in diameter or larger. The present invention may be used to activate tooth whitening agents through an optical component on both upper and lower tooth arches simultaneously or one tooth at a time. The present invention may be used as a source of illumination in the oral cavity, in medical surgery, for machine vision, or other.

Tissue Interactions Include Indications For Use In:
Open and Endoscopic Surgery; light assisted procedures provide a level of surgical precision not available with other mechanical means and where the benefits of the hemostasis effect of the light is realized.
Photodynamic Therapy and Biostimulation; relatively low light levels are used to alter or otherwise stimulate living tissue in therapeutically useful ways.
Pain Control; light induced analgesia and nerve stimulation therapy.
Tissue welding and fusion; seals biological tissue without sutures.

Primary Light-Tissue Interactions:
Photothermal where light is absorbed by tissue and converted to heat energy or where water or other molecules absorb light energy and heat tissues indirectly.

Other Light-Tissue Interactions:
Photochemical/Photodynamic; light absorbing molecules result in a chemical reaction with tissue or the formulation of a biochemically reactive singlet oxygen molecule.
Biostimulation employs relatively low light levels to stimulate healing of tissue and pain relief.

The present invention improves upon the use lasers for cosmetic and surgical soft tissue procedures through the use of a broad spectrum light source that more closely matches the absorptive region of the target tissues compared to monochromatic lasers. Laser procedures with prior FDA clearance include soft tissue curettage, removal of diseased and inflamed tissue affected by bacteria from the periodontal pocket, sulcular debridement in the periodontal pocket, cosmetic gingival contouring, gingival troughing, crown lengthening, treatment of herpetic lesions and aphthous ulcers, and other indications.

The present invention is of unique construction that allows the delivery of appropriate levels of electromagnetic energy to target tissue through an optical component less than 1 mm in diameter or larger while still retaining the benefits of the light source for the photo initiation of dental light cure resins or other light cure materials, the activation of tooth whitening agents, and as a general illumination source.

The xenon lamp used in the light source emits electromagnetic energy over a spectrum of 380 nm to 1200 nm. There are peaks of energy at approximately 470 nm, 780 nm, 830 nm, 900 nm, 950 nm, 980 nm, etc. that are typical of xenon lamps. The spectral peaks of a xenon lamp are different than halogen, metal halide, or mercury vapor although all of these produce electromagnetic energy over roughly the same spectral range.

By using one or more bandpass filters it is possible to control the wavelengths so that only those that are desired for a particular procedure are transmitted from the final optical component.

The multi-purpose light source can be used for dental curing, tooth whitening, treatment of biologic tissues, illumination of the general oral cavity, illumination inside of a tooth cavity or root cannel, transillumination of the tooth for caries and crack detection, and fluorescence of bacterial and other pathogens.

1) For dental curing, a filter transmits electromagnetic energy from 380 nm-520 nm and blocks other wavelengths. The energy is focused into a flexible light guide of approximately 2 mm-5 mm comprised of multiple fiber-optic strands or a single liquid filled core. A rigid fused rod, clad rod, or optical acrylic end tip is used to direct the energy to the treatment area.

2) For tooth whitening, a filter transmits electromagnetic energy from 380 nm-520 nm and blocks other wavelengths. The energy is focused into a flexible light guide of approximately 2 mm-5 mm comprised of multiple fiber-optic strands or a single liquid filled core. A rigid fused rod, clad rod, or optical acrylic end tip is used to direct the energy to the treatment area. In addition, Den-Mat has a patented device that directs energy to both upper and lower tooth arches simultaneously.

3) For treatment of biologic tissues, a filter transmits electromagnetic energy from approximately 650 nm-1200 nm and blocks other wavelengths. It is possible that wavelengths from 380 nm-650 nm may be used but is not anticipated at this time. The energy is focused into a flexible light guide of approximately 1 mm-3 mm comprised of multiple fiber-optic strands or a single liquid filled core. An attachment at the distal end of the flexible light guide diverges or collimates the light into and end tip that is 100 micron to 600 micron in diameter and constructed of a single optical fiber, glass rod, or optical acrylic. This end tip is then used in a contact or non-contact mode with biologic tissue. It is anticipated that electromagnetic energy measured at the distal face of the 100 micron to 600 micron end tip will be from 0-5 watts of power. The operator will adjust the output level to achieve the desired effect on the tissue. It is anticipated that the wavelengths available will have an effect on soft tissues containing water and blood but will not have an effect on hard tissue such as tooth enamel and bone. It is anticipated that the use of a diverging or collimating lens set, an optical taper, or other means may be desired to control the geometry of the light prior to entering the flexible light guide or at the attachment on the distal end prior to the light entering the final working end tip.

4) For illumination of the general oral cavity, a filter transmits electromagnetic energy from approximately 400 nm-700 nm (the visible spectrum). Further, an additional filter transmits only between 520 nm-700 nm (to prevent photo curing of dental resins). A light dispersion device is attached to the distal end of a flexible light guide. This device fits into the patients mouth and acts as a bite block to keep the mouth open.

5) For illumination of a tooth cavity or root cannel, light is filtered as above and transmitted through a single fiber attached to a 1 mm-3 mm flexible light guide.

6) For transillumination an attachment on the end of a 1 mm-3 mm flexible light guide emits light into 2 sides of a tooth. When the inside of the tooth is illuminated caries and cracks show up as dark areas.

7) For fluorescence of bacterial and other pathogens when light of one wavelength illuminates selected bacteria a different wavelength is emitted.

Delivery of light energy through an optical component less than 1 mm in diameter or larger is necessary for surgical precision on target tissue. This is especially necessary for procedures within the periodontal pocket and other confined areas. The ability to deliver appropriate levels of electromagnetic energy to target tissue through an optical component less than 1 mm in diameter or larger while still retaining the benefit of the light source for other uses is made possible through the unique design of optical and other component designs contained in the light source. The light source, in total, and the unique components individually make up the present invention.

Individual components that comprise the present invention include but are not limited to:

1) xenon lamp with unique reflector geometry and arc gap

2) light guide and hand piece with optical taper and focusing optics

3) micro-taper tip less than 1 mm diameter

4) filter changer with Visible and IR band pass filters

5) variable switching power supply with self-monitoring feedback loop

A xenon lamp with unique reflector geometry and arc gap focuses maximum light energy into a small 2 mm-3 mm diameter light guide. Lamp may be doped to increase useful wavelengths.

Light guide and hand piece with optical taper and focusing optics, 2 mm-3 mm in diameter or smaller, includes (if required) optical taper and other focusing optics to collect and collimate light emitted from the lamp. Hand piece is of unique design to hold micro-taper tip as well as other interchangeable optical devices.

Micro-taper tip(s) constructed of single or multiple glass fibers, a single piece of clad rod, or molded plastic of different sizes and shapes with distal end less than 1 mm or larger.

Filter changer between xenon lamp and light guide that electronically changes filters, as selected by the operator, depending on the procedure the light is being used for.

Variable switching power supply with a self-monitoring feedback loop allows the operator to select power levels. Feedback loop monitors the light output and self adjusts current to the lamp to maintain light output at selected levels.

### EXAMPLE 1

An existing xenon lamp similar to that of the present invention was coupled with a 3 mm fiberoptic bundle and a 3 mm to 0.70 mm micro-taper tip. An IR transmitting filter transmitting above>577 nm with a peak at 824 nm at was placed between the lamp and the light guide. Light output of approximately 3 watts was achieved.

The distal end of the micro-taper was placed in contact with a piece of cooked ham. The effect was similar to that of using the Biolase Diolase soft tissue laser. The distal end of the micro-taper tip was carbonized and then place in contact with a piece of cooked ham. An immediate charring effect was noted with vaporization (popping and smoke) occurring. Sliding the end of the tip slowly across the sample caused a troughing or “cutting” effect.

Soft tissue lasers are typically be used between 1-2 watts continuous wave mode. The xenon lamp and more efficient filter of the present invention are expected to produce 2-3 times the light output or somewhere between 6 and 9 watts. This is more total light output than other soft tissues lasers on the market and the light energy is more efficient because it better matches the absorptive regions of the targets.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of devices would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What we claim is:

1. A multi-use light comprising:
a light source;
a light guide;
a filter changer; and
a power supply.

2. The multi-use light source of claim 1 wherein the light source is a xenon lamp.

3. The multi-use light source of claim 1 wherein the filter changer comprises a visible band pass filter.

4. The multi-use light source of claim 1 wherein the filter changer comprises an IR band pass filter.

5. The multi-use light source of claim 1 wherein the filter changer comprises a combination of a visible band pass filter and an IR band pass filter.

6. A method of modifying the output of a light source comprising the following steps:
providing a source of light;
guiding the light;
modifying the wavelength of the light by passing through a filter; and
directing the modified light to a desired target.

7. The method of claim 6, wherein the light source comprises a xenon lamp, a visible band pass filter, and an IR band pass filter.

8. A multi-use light comprising:
a light source;
a means for guiding light;
a means for filtering light; and
a means for supplying power.

9. The light source of claim 8 wherein the light source is a xenon lamp.

10. The light source of claim 8 wherein the filter is a visible band pass filter.

11. The light source of claim 8 wherein the filter is an IR band pass filter.

12. The light source of claim 8 wherein the filter changer comprises a combination of a visible band pass filter and an IR band pass filter.

13. The light source of claim 8 further comprising a means for changing filters.

14. A method of treating tissue comprising
providing a source of light;
guiding the light;
modifying the wavelength of the light by passing through a filter; and
directing the modified light to a desired tissue.

15. The method of claim 14, wherein the light source comprises a xenon lamp, a visible band pass filter, and an IR band pass filter.
